(19) United States
(12) Patent Application Publication
(10) Pub. No.: US 2018/0369538 A1
(43) Pub. Date: Dec. 27, 2018

(54) CATHETER

(71) Applicants: THE ASAN FOUNDATION, Seoul (KR); AJOU UNIVERSITY INDUSTRY-ACADEMIC COOPERATION FOUNDATION, Suwon-si (KR); UNIVERSITY OF ULSAN FOUNDATION FOR INDUSTRY COOPERATION, Ulsan (KR)

(72) Inventors: Deok Hee LEE, Seoul (KR); Ji Hoon SHIN, Seoul (KR); Seon Moon HWANG, Bucheon-si (KR); Tae Il KIM, Seoul (KR); Ga Young LEE, Seoul (KR); Eun Joung KIM, Gunpo-si (KR); Jin Ho YOON, Namyangju-si (KR); Seng Yong CHUN, Seoul (KR); Ki Dong PARK, Seoul (KR); Yun Ki LEE, Seongnam-si (KR)

(73) Assignees: The Asan Foundation, Seoul (KR); AJOU University Industry-Academic Cooperation Foundation, Suwon-si (KR); University of Ulsan Foundation for Industry Cooperation, Ulsan (KR)

(21) Appl. No.: 15/780,834

(22) PCT Filed: Nov. 4, 2016
(86) PCT No.: PCT/KR2016/012674

§ 371 Date: Jun. 1, 2018

(30) Foreign Application Priority Data
Dec. 4, 2015 (KR) 10-2015-0172688

(51) Int. Cl. A61M 25/00; A61L 29/14

(52) U.S. Cl. CPC A61M 25/0045; A61M 25/0021; A61L 29/145; A61L 29/146

(57) ABSTRACT

Provided is a catheter to be inserted into an object. The catheter includes a main body having a tubular shape and an expansion layer formed on an outer circumferential surface of the main body, and the expansion layer contains an expandable material which is expanded by contact with a liquid.

### TECHNICAL FIELD

The present disclosure relates to a catheter in which expansion of an outer diameter can be induced.

### BACKGROUND

In the case where a catheter is inserted into a human blood vessel or alimentary canal and left in for the purpose of treatment of disease (for example, central venous catheter insertion (C-line insertion)), the blood vessel may be damaged during the insertion of the catheter or loosening of a part of a vascular membrane in contact with the catheter may cause persistent bleeding with a high frequency.

After the catheter is inserted and left in, an external force applied to the catheter or a movement of an object may cause the formation of a gap in an area where the catheter is inserted, and, thus, body fluids such a blood may leak out.

The background technology of the present disclosure is disclosed in Korean Patent Laid-open Publication No. 10-2005-0019768.

### DISCLOSURE OF THE INVENTION

### Problems to be Solved by the Invention

In view of the foregoing, the present disclosure provides a catheter of which an outer diameter can be increased to compensate for a gap in an area where the catheter is inserted.

Further, the present disclosure provides a catheter which can suppress the loss of an expandable material of an expansion layer that encases a main body of the catheter.

Furthermore, the present disclosure provides a catheter which can control the speed of expansion of an expandable material.

Moreover, the present disclosure provides a catheter which can suppress expansion of an expandable material in a longitudinal direction of the catheter.

However, problems to be solved by the present disclosure are not limited to the above-described problems. There may be other problems to be solved by the present disclosure.

### Means for Solving the Problems

As a technical means for solving the above-described technical problems, a catheter according to an exemplary embodiment of the present disclosure includes a main body having a tubular shape and an expansion layer formed on an outer circumferential surface of the main body, and the expansion layer may contain an expandable material which is expanded by contact with a liquid.

According to an example of the present exemplary embodiment, the catheter may further include a mask layer formed on the expansion layer.

According to an example of the present exemplary embodiment, the mask layer may include a porous member in which multiple holes are formed.

According to an example of the present exemplary embodiment, the holes may have a size that allows the liquid to pass through but does not allow the expandable material to pass through while the catheter is inserted in an object.

According to an example of the present exemplary embodiment, the mask layer may include multiple slot parts whose opening size is controllable to control a speed of expansion of the expandable material by adjusting the amount of a liquid introduced into the expansion layer.

According to an example of the present exemplary embodiment, the mask layer may include a first mask layer in which multiple first slots are formed and which is formed on the expansion layer and a second mask layer in which multiple second slots are formed and which is formed on the first mask layer, and the multiple slot parts are controllable in size depending on the degree of overlap between the multiple first slots and the multiple second slots.

According to an example of the present exemplary embodiment, the mask layer may be detachably attached.

According to an example of the present exemplary embodiment, the expandable material may include hydrogel.

According to an example of the present exemplary embodiment, the catheter may further include a pair of constraining members respectively arranged on both ends of the expansion layer in a longitudinal direction of the main body and configured to suppress expansion of the expandable material in the longitudinal direction of the main body.

According to an example of the present exemplary embodiment, the constraining member may be formed into a ring shape that encases the outer circumferential surface of the main body.

According to an example of the present exemplary embodiment, at least one of the constraining members may have a taper on an access surface facing the object to facilitate insertion into the object.

The above-described exemplary embodiments are provided by way of illustration only and should not be construed as liming the present disclosure. Besides the above-described exemplary embodiments, there may be additional exemplary embodiments described in the accompanying drawings and the detailed description.

### Effects of the Invention

According to the above-described means for solving the problems, the expansion layer containing the expandable material is formed on the outer circumferential surface of the main body, and, thus, a space which may cause loosening between the catheter inserted in the object and a vascular membrane can be strengthened by the expandable material. Therefore, it is possible to suppress bleeding such as a leak of a small amount of blood.

Further, according to the above-described means for solving the problems, the mask layer including multiple holes having a size that does not allow the expandable material to pass through is laminated on the expansion layer, and, thus, it is possible to suppress the loss of the expandable material.

Furthermore, according to the above-described means for solving the problems, the amount of the liquid introduced into the expansion layer is adjusted by adjusting the size of the first slots in the first mask layer and the size of the second slots in the second layer which overlap with each other, and, thus, it is possible to control the speed of expansion of the expandable material.

Moreover, according to the above-described means for solving the problems, the constraining members are respectively arranged on the both ends of the expansion layer in the longitudinal direction of the main body, and, thus, it is possible to suppress expansion of the expandable material in the longitudinal direction of the catheter.

Further, the effects to be achieved by the present disclosure are not limited to the above-described effects. Although not described herein, other effects to be achieved by the present disclosure can be clearly understood by those skilled in the art from the following description.

### BRIEF DESCRIPTION OF THE DRAWINGS

FIG. 1 is a cross-sectional view of a catheter including a porous member according to an exemplary embodiment of the present disclosure;

FIG. 2 is a cross-sectional view of a catheter including multiple slot parts according to an exemplary embodiment of the present disclosure;

FIG. 3A and FIG. 3B are schematic partial plan views provided to explain the degree of opening of slot parts (the degree of exposure of an expansion layer to a liquid) in the catheter illustrated in FIG. 2; and

FIG. 4 is a schematic cross-sectional view provided to explain constraining members according to an exemplary embodiment of the present disclosure.

### BEST MODE FOR CARRYING OUT THE INVENTION

Hereinafter, embodiments of the present disclosure will be described in detail with reference to the accompanying drawings so that the present disclosure may be readily implemented by those skilled in the art. However, it is to be noted that the present disclosure is not limited to the embodiments but can be embodied in various other ways. In drawings, parts irrelevant to the description are omitted for the simplicity of explanation, and like reference numerals denote like parts through the whole document.

Through the whole document, the term “connected to” or “coupled to” that is used to designate a connection or coupling of one element to another element includes both a case that an element is “directly connected or coupled to” another element and a case that an element is “electronically connected or coupled to” another element via still another element.

Through the whole document, the term “on” that is used to designate a position of one element with respect to another element includes both a case that the one element is adjacent to the other element and a case that any other element exists between these two elements.

Further, through the whole document, the term “comprises or includes” and/or “comprising or including” used in the document means that one or more other components, steps, operation and/or existence or addition of elements are not excluded in addition to the described components, steps, operation and/or elements unless context dictates otherwise.

Through the whole document, the term “about or approximately” or “substantially” is intended to have meanings close to numerical values or ranges specified with an allowable error and intended to prevent accurate or absolute numerical values disclosed for understanding of the present disclosure from being illegally or unfairly used by any unconscionable third party. Through the whole document, the term “step of” does not mean “step for”.

Through the whole document, the term “unit” or “means” includes a unit implemented by hardware, a unit implemented by software, and a unit implemented by both of them. One unit may be implemented by two or more pieces of hardware, and two or more units may be implemented by one piece of hardware.

Through the whole document, a part of an operation or function described as being carried out by a terminal, apparatus or device may be carried out by a server connected to the terminal, apparatus or device. Likewise, a part of an operation or function described as being carried out by a server may be carried out by a terminal, apparatus or device connected to the server.

A catheter according to an exemplary embodiment of the present disclosure can be inserted into an object, and the object used herein may refer to a living body such as a human body.

Hereinafter, a catheter according to an exemplary embodiment of the present disclosure will be described.

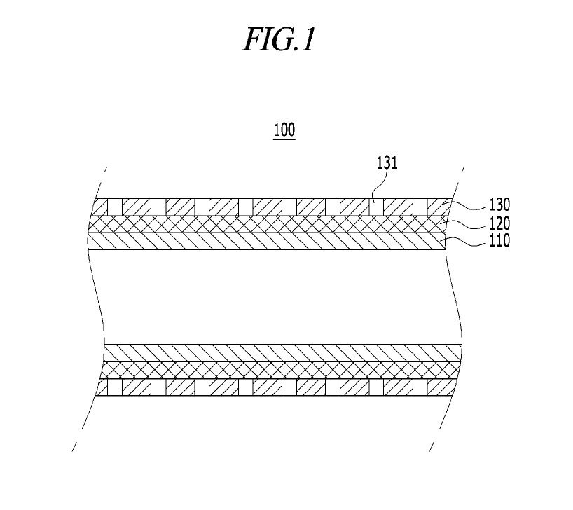

FIG. 1 is a cross-sectional view of a catheter according to an exemplary embodiment of the present disclosure.

As illustrated in FIG. 1, a catheter 100 of the present disclosure may include a main body 110 and an expansion layer 120.

The main body 110 may be formed into a tubular shape. The main body can be inserted into an organ having a cavity or lumen. Further, the main body 110 may be formed of a material including at least one of polyurethane and polyvinyl chloride (PVC) resin.

The expansion layer 120 may be formed on an outer circumferential layer of the main body 110. Further, the expansion layer 120 may contain an expandable material which is expanded by contact with a liquid. For example, the liquid may include body fluids such as blood, lymph, bile, and the like.

Further, the expandable material may be hydrogel. Specifically, the hydrogel included in the expandable material of the present disclosure may be hydrogel which is characterized by expansion of 10% or more in volume by reaction with moisture when being in contact with a liquid such as blood (body fluid) within an object (human body).

That is, in the case where the main body 100 is inserted into the object (human body), the expandable material may be exposed to a liquid and expanded by reaction with moisture and thus may increase an outer diameter of the catheter 100. Therefore, even if a gap is formed between the catheter 100 and an area of the object where the catheter 100 is inserted, the gap may be filled by expansion of the expandable material. Therefore, it is possible to suppress leakage of the liquid, such as blood (body fluid), through the gap. Further, the expandable material can suppress secondary infections which may occur due to the leakage of the liquid.

Meanwhile, an expansion rate of the expandable material in the catheter 100 may be set differently depending on the purpose of use. For example, as for the purpose of tract dilatation, it is desirable to gradually expand the outer diameter of the catheter for a day or two and expand the outer diameter of the catheter to the max (from 10 French to 20 French) in about a week. For another example, as for a central line catheter, it may be desirable to expand the outer diameter of the catheter to the max within an hour of insertion. As for a pigtail catheter, it may be desirable to gradually expand the diameter of the catheter to a predetermined maximum diameter over a period of insertion in the object.

Further, the speed of increase (expansion) of the outer diameter of the catheter may be adjusted by adjusting components of the expandable material contained in the expansion layer. For example, as for hydrogel included in the expandable material, the expansion rate can be set for the purpose of use by properly adjusting components of the hydrogel. The setting of the expansion rate at the time of contact with moisture by adjusting components of the hydrogel is known to those skilled in the art, and, thus, a more detailed explanation thereof will be omitted.

For yet another example, although will be described later, a mask layer 130 that encases the expansion layer 120 basically blocks a liquid introduced into the expansion layer 120, and slot parts whose opening size is controllable are formed in the mask layer 130, and, thus, the amount of the liquid introduced into the expansion layer can be adjusted depending on the degree of opening of the slot parts. Therefore, it is possible to adjust the speed of expansion of the expandable material and the speed of increase of the outer diameter of the catheter.

The catheter 100 according to an exemplary embodiment of the present disclosure may include the mask layer 130.

The mask layer 130 may be laminated on the expansion layer 120 as illustrated in FIG. 1.

It is possible to suppress exposure of the expansion layer 120 to a liquid by using the mask layer 130. Further, the mask layer 130 may be detachably attached to the catheter 100. That is, the mask layer 130 that encases the expansion layer 120 can be removed when the expansion layer 120 needs to be exposed to the liquid, and, thus, the expandable material can be expanded at the right time (for example, the time when bleeding starts due to loosening of a vascular membrane).

Further, the mask layer 130 may include a porous member in which multiple holes 131 are formed.

In this case, it should be understood that the mask layer 130 does not suppress exposure of the expansion layer 120 to the liquid but functions as a selective net that allows the expansion layer 120 to be exposed to the liquid through the multiple holes 131 and suppresses the loss of the expandable material constituting the expansion layer 120.

The multiple holes 131 have a size that allows the liquid to pass through but does not allow the expandable material to pass through while the catheter 100 is inserted in the object. For example, while the catheter 100 is inserted in the object, various external forces (for example, skin pressure, blood pressure, external forces of other people, etc.) may be applied to the catheter 100. The present inventors anticipated that a part of the expandable material of the expansion layer 120 would be separated and lost by such external forces and then would remain within the object. To solve this problem, the present inventors have attempted to apply a porous member which allows a liquid to pass through but does not allow the expandable material to pass through for the mask layer 130. That is, desirably, the multiple holes 131 may have a size that does not allow the expandable material to pass through (but selectively allows a liquid to pass through) within a predetermined range of pressure.

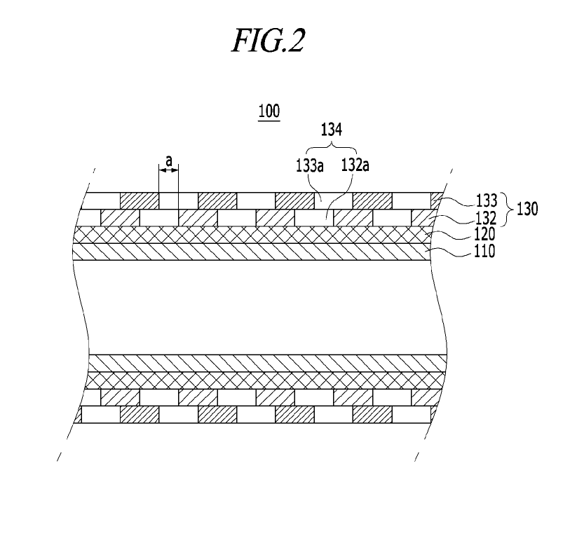

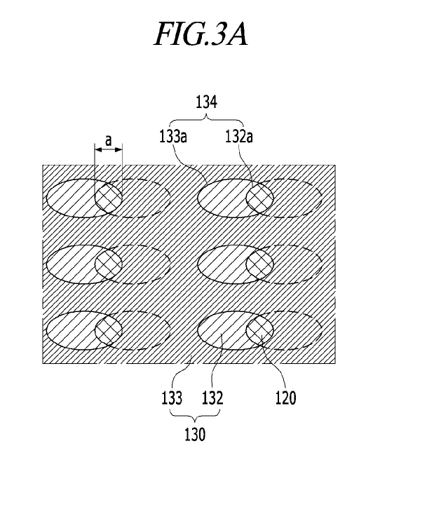

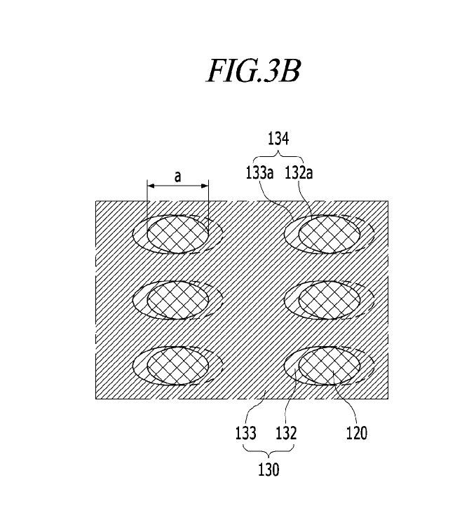

FIG. 2 is a cross-sectional view of a catheter including multiple slot parts according to an exemplary embodiment of the present disclosure, and FIG. 3A and FIG. 3B are schematic partial plan views provided to explain the degree of opening of slot parts (the degree of exposure of an expansion layer to a liquid) in the catheter illustrated in FIG. 2.

As illustrated in FIG. 2 and FIG. 3A and FIG. 3B, the mask layer 130 may include multiple slot parts 134.

An opening size a of the multiple slot parts 134 can be adjusted to control the speed of expansion of the expandable material by adjusting the amount of the liquid introduced into the expansion layer 120.

For example, if the opening size a of the multiple slot parts 134 is increased, the degree of exposure of the expansion layer 120 is increased accordingly, and, thus, the amount of the liquid in contact with the expansion layer 120 is also increased. Therefore, the speed of expansion of the expandable material can be adjusted rapidly (see FIG. 3A). For another example, if the opening size a of the multiple slot parts 134 is decreased, the degree of exposure of the expansion layer 120 is decreased accordingly, and, thus, the amount of the liquid in contact with the expansion layer 120 is also decreased. Therefore, the speed of expansion of the expandable material can be adjusted slowly.

Specifically, referring to FIG. 2, the mask layer 130 may include a first mask layer 132 and a second mask layer 133.

The first mask layer 132 is formed on the expansion layer 120 and may include multiple first slots 132a. Further, the second mask layer 133 is formed on the first mask layer 133 and may include multiple second slots 133a.

In an exemplary embodiment of the dual-structure mask layer as described above, the multiple slot parts 134 may include one of the multiple first slots 132a and one of the multiple second slots 133a corresponding thereto. That is, the opening size a of the multiple slot parts 134 may be adjusted depending on the degree of overlap between the first slot 132a and the second slot 133a.

For example, referring to FIG. 3A, as an overlap area a between the first slot 132a and the second slot 133a corresponding thereto is increased, the slot part 134 may be gradually opened and the expansion layer 120 positioned therein may be more exposed to the outside. Meanwhile, referring to FIG. 3B, as the overlap area a between the first slot 132a and the second slot 133a corresponding thereto is decreased, the slot part 134 may be gradually closed and the expansion layer 120 positioned therein may be less exposed to the outside.

In this regard, a method of adjusting the degree of overlap a between the first slot 132a in the first mask layer 132 and the second slot 133a in the second mask layer 133 can be implemented in various ways.

For example, while the catheter is inserted in the object, the catheter may be provided in order for a part of the second mask layer 133 or a connection member connected to the second mask layer 133 to be exposed to the outside of the object. In this case, a medical team may manually manipulate the part exposed to the outside of the object, and, thus, the opening size a of the slot parts 134 may be adjusted. More specifically, if the second mask layer 133 is pulled to the right in the drawing while the slot parts 134 are opened as illustrated in FIG. 3B, the second mask layer 133 is moved by a predetermined amount to the right as illustrated in FIG. 3A, and, thus, the opening size a of the slot parts 134 can be increased. However, the method of adjusting the opening size a is not limited thereto. Further, the porous mask layer 130 illustrated in FIG. 1 and the slot-type mask layer 130 in which the slots can be controlled in size as illustrated in FIG. 2, FIG. 3A and FIG. 3B may be applied together to the catheter 100 of the present disclosure.

In other words, the mask layer 130 of the present disclosure may be porous with the small-sized holes 131 that suppresses the loss of the expandable material and allows the introduction of the liquid and may also include the multiple slot parts 134 of which the opening size a can be adjusted if necessary. For example, the speed of expansion of the expandable material may be the lowest when the liquid is introduced only through the holes 131 while the slot parts 134 are fully closed. In this case, as the slot parts 134 are gradually opened, the speed of expansion of the expandable material can be increased, and when the first slot 132a and the second slot 133a are overlapped as much as possible and the slot parts 134 are opened to the max, the speed of expansion of the expandable material can be increased to the max.

According to the present disclosure, the porous structure and the variable slot structure are combined organically, and, thus, it is possible to suppress the loss of an expandable material to a certain extent or more and also possible to increase an outer diameter of the catheter by introducing a liquid. Further, it is possible to more accurately control the time to maximize the outer diameter of the catheter by adjusting the speed of introducing the liquid depending on the purpose of use of the catheter.

Also, desirably, the mask layer 130 may be formed of a material which can be elastically expanded according to expansion of the expansion layer 120 positioned therein. For example, the mask layer 130 may be formed of an elastic material.

Further, the catheter 100 of the present disclosure may include constraining members 140.

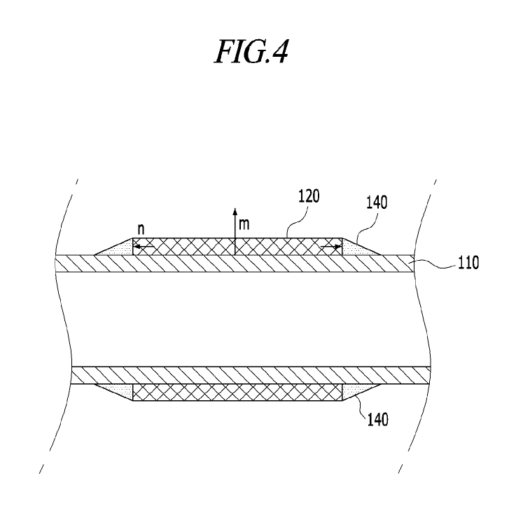

FIG. 4 is a schematic cross-sectional view provided to explain constraining members according to an exemplary embodiment of the present disclosure.

Referring to FIG. 4, the constraining members 140 may be respectively arranged on the both ends of the expansion layer 120 in a longitudinal direction of the main body 110. For example, the constraining member 140 may be formed into a ring shape that encases an outer circumferential surface of the main body 110. However, the constraining member 140 is not limited to the ring shape, and may have any shape including the ring shape as long as it can encase the outer circumferential surface of the main body 110.

Referring to FIG. 4, the pair of constraining members 140 can suppress expansion of the expandable material in a longitudinal direction n of the main body 110. Since the constraining members 140 suppress the expansion of the expandable material in the longitudinal direction of the main body 110, the volume of the expandable material may be increased in a radial direction m of the catheter 100. That is, the constraining members 140 may restrict the expansion of the expandable material in the longitudinal direction of the main body 110 and thus induce the volume expansion of the expandable material to fully function as expansion of the outer diameter of the catheter 100.

Further, referring to FIG. 4, at least one of the constraining members 140 may have a taper on an access surface facing the object to facilitate insertion into the object.

Since the constraining member 140 has the slanted taper on the access surface, the catheter 100 can be more easily inserted or removed when being inserted into the object or separated from the object.

The above description of the present disclosure is provided for the purpose of illustration, and it would be understood by those skilled in the art that various changes and modifications may be made without changing technical conception and essential features of the present disclosure. Thus, it is clear that the above-described embodiments are illustrative in all aspects and do not limit the present disclosure. For example, each component described to be of a single type can be implemented in a distributed manner. Likewise, components described to be distributed can be implemented in a combined manner.

The scope of the present disclosure is defined by the following claims rather than by the detailed description of the embodiment. It shall be understood that all modifications and embodiments conceived from the meaning and scope of the claims and their equivalents are included in the scope of the present disclosure.

We claim:

1. A catheter to be inserted into an object, comprising:
a main body having a tubular shape; and
an expansion layer formed on an outer circumferential surface of the main body,
wherein the expansion layer contains an expandable material which is expanded by contact with a liquid.

2. The catheter of claim 1, further comprising:
a mask layer formed on the expansion layer.

3. The catheter of claim 2,
wherein the mask layer includes a porous member in which multiple holes are formed.

4. The catheter of claim 3,
wherein the holes have a size that allows the liquid to pass through but does not allow the expandable material to pass through while the catheter is inserted in the object.

5. The catheter of claim 2,
wherein the mask layer includes multiple slot parts whose opening size is controllable to control a speed of expansion of the expandable material by adjusting the amount of a liquid introduced into the expansion layer.

6. The catheter of claim 2,
wherein the mask layer includes:
a first mask layer in which multiple first slots are formed and which is formed on the expansion layer; and
a second mask layer in which multiple second slots are formed and which is formed on the first mask layer, and
wherein the multiple slot parts are controllable in size depending on the degree of overlap between the multiple first slots and the multiple second slots.

7. The catheter of claim 2,
wherein the mask layer is detachably attached.

8. The catheter of claim 1,
wherein the expandable material includes hydrogel.

9. The catheter of claim 1, further comprising:
a pair of constraining members respectively arranged on both ends of the expansion layer in a longitudinal direction of the main body and configured to suppress expansion of the expandable material in the longitudinal direction of the main body.

10. The catheter of claim 9,
wherein the constraining member is formed into a ring shape that encases the outer circumferential surface of the main body.

11. The catheter of claim 9,
wherein at least one of the constraining members has a taper on an access surface facing the object to facilitate insertion into the object.
